(19) United States
(12) Patent Application Publication
(10) Pub. No.: US 2024/0174970 A1
(43) Pub. Date: May 30, 2024

(54) METHOD FOR OPERATING A BIOPROCESS INSTALLATION

(71) Applicant: Sartorius Stedim Biotech GmbH, Goettingen (DE)

(72) Inventors: Martin Saballus, Neu-Eichenberg (DE); Lucas Nik Reger, Goettingen (DE); Markus Kampmann, Dortmund (DE); Jens-Christoph Matuszczyk, Goettingen (DE)

(21) Appl. No.: 18/281,159

(22) PCT Filed: Mar. 10, 2022
(86) PCT No.: PCT/EP2022/056126

§ 371 Date: Sep. 8, 2023

(30) Foreign Application Priority Data
Mar. 10, 2021 (EP) 21161867.3

(51) Int. Cl. C12M 1/36; C12M 1/00; C12M 1/26; C12M 1/34

(52) U.S. Cl. CPC C12M 41/48; C12M 29/26; C12M 33/10; C12M 41/30

(57) ABSTRACT

A method for operating a bioprocess installation with an electronic process control and at least one bioprocess unit, wherein the bioprocess unit comprises a cell broth source with a first receptacle for cell broth including cultivation media and cells, establishing a culture environment for cell cultivation and/or bio production, wherein the bioprocess unit comprises a clarification setup with a centrifuge for the clarification of the cell broth by centrifugation, with a liquid pumping arrangement assigned to the centrifuge and with a liquid network with a number of liquid lines communicating with the liquid pumping arrangement, wherein out of a first culture environment established by the first receptacle, the cell broth is transfered to the centrifuge via the liquid network, which centrifuge is operated in a forward operation for cell separation and/or cell washing and in a backward operation for cell discharging.

### CROSS-REFERENCE TO RELATED APPLICATIONS

This application is a national stage application under 35 U.S.C. 371 of International Patent Application Serial No. PCT/EP2022/056126, entitled “Method for Operating a Bioprocess Installation,” filed Mar. 10, 2022, which claims priority from European Patent Application No. EP 21161867.3, filed Mar. 10, 2021, the disclosure of which is incorporated herein by reference.

### FIELD OF THE TECHNOLOGY

Various embodiments relate to a method for operating a bioprocess installation, a bioprocess installation with an electronic process control and at least one bioprocess unit, an electronic process control of the bioprocess installation and use of a pre-mounted liquid network product.

### SUMMARY

The term “bioprocess” presently represents any kind of biotechnological processes, in particular biopharmaceutical processes. An example for such bioprocess is the use of a bioreactor to cultivate microorganisms or mammalian cells under given conditions, wherein a cell broth is transferred from the bioreactor to a downstream process.

The method in question for operating a bioprocess installation may be applied in various fields of biotechnology. High efficiency in this field has been driven by the increasing de-mand for biopharmaceutical drugs. The efficiency in this sense is regarding not only the cost-effectiveness of the components to be used but also the controllability of the processes connected thereto. The method in question serves for establishing a culture environment for cell cultivation and/or bioproduction, followed by clarification of a cell broth by centrifugation and subsequent transfer of discharged cells to a second culture environment via the recycling line for subsequent cell cultivation and/or bioproduction. Possible products can be for instance biopharmaceutical products, in particular proteins, such as growth factors, hormones, enzymes, antibodies, antibody derivates, or the like.

The known method for operating a bioprocess installation (EP 2 310 486 B1), which is the starting point for various embodiments, makes use of a centrifuge with a number of centrifuge chambers, which are each assigned a chamber inlet and a chamber outlet. The method for operating a bioprocess installation makes also use of a liquid pumping arrangement assigned to the centrifuge and a liquid network for the transport of the liquid, which is based on the cell broth to be clarified. After the clarification process, the cells can be re-transferred into the same bioreactor leading to a backmixing of cells and media, including dissolved substances, e.g. toxins. Finally, the method for operating a bioprocess installation makes use of a process control for controlling at least the centrifuge and the liquid pumping arrangement. The known method as such is running in a highly efficient manner. However, the resulting combination is of restricted efficiency in view of product yield, process flexibility and adjustability in the mechanical setup as well as in the controllability of the overall process.

It is an object of some embodiments to provide a method for operating a bioprocess installation, which increases the bioprocess efficiency and productivity, with as little effort as possible.

The above-noted problem is solved for a method for operating a bioprocess installation with the features as described herein.

The general concept underlying various embodiments is based on a transfer of at least a part of the discharged cells to a second culture environment, that is different from the first culture environment.

The term “culture environment” presently means the complex influence of the entirety of all physical, chemical and/or biological parameters, such as temperature, pH and nutrition concentrations, that act upon a cultivated organism and ultimately determine its physiology and survival.

The above is facilitated by a liquid network comprising a recycling line and by a backward operation of a centrifuge. In the second culture environment, which is different from the first culture environment, the recycled cells can be used for subsequent cell cultivation and/or bioproduction. The second culture environment, being different from the first culture environment, gives the basis for an improvement of the subsequent culture environment. First of all, this enables to influence, such as to optimize, the culture environment leading to influ-enced, such as optimized and/or accelerated, cultivation of recycled cells and/or bioproduction by these recycled cells. Second, this allows for elongated cultivation by recycling harvested viable cells, since no seed train needs to be generated for a subsequent bioprocess. Third, this setup allows for an entire removal of consumed media, preventing a backmixing of consumed media with fresh media, including cellular produced inhibitors. This means, that no continuous media exchange is required during cultivation, leading to a reduced media consumption, higher product concentrations, as well as increased productivity, which is not only cost-efficient but also enables a simplified and more efficient control of the overall process. Eventually, this leads to reduced process times and cultivation costs with minimal effort, leading to an increased bioprocess efficiency. Furthermore, this simplified setup allows for an adaption to existing bioprocesses, independent from the process platforms and cell lines used in the respective bioprocess.

In detail, it is proposed, that the liquid network comprises a recycling line and that in the backward operation, at least part of the discharged cells is being transferred to a second culture environment different from the first culture environment via the recycling line for subsequent cell cultivation and/or bioproduction.

The term “recycling line” relates to a liquid line designed for the re-transfer and hence re-use of discharged cells, thus making the cells accessible for subsequent cell cultivation and/or bioproduction.

Various embodiments provide the possible differences between the first and second culture environment and enables for an increased flexibility regarding the choice of the respective entity, liquid properties and/or culture environment conditions of the first and second culture environment.

According to some embodiments, the second culture environment can provide culture environment conditions favouring cell growth and/or bioproduction, wherein, alternatively or in addition, the second culture environment is the first culture environment, after having exchanged the cultivation media. All of this allows for a boost in cell physiology prior to bioproduction. An exchange of the cultivation media prevents a backmixing of consumed media and fresh media.

Various embodiments are directed to the involved receptacles, which allow for an increased flexibility regarding the choice of the second receptacle.

Various embodiments are directed to the transfer of the cell broth from the second receptacle to the first receptacle after an intermediate phase. This allows nourishing the cells before they are used for subsequent cell cultivation and/or bioproduction.

According to various embodiments, a termination condition has to be fulfilled with respect to the liquid level in the first receptacle, before the cell broth is transferred from the second to the first receptacle. This is another measure to prevent backmixing of consumed and fresh media.

Various embodiments are directed to the bioprocess units being cascaded. This cascade offers the advantage that allows for a continuous recycling of cells and their re-use in a subsequent bioprocess.

According to various embodiments, a startup phase is initiated in the second culture environment in order to establish conditions for cell cultivation and/or bioproduction. This leads to an increased ratio of viable cells and hence to an increased productivity of the bioprocess.

In various embodiments, the properties of the cell washing step and cell discharging step are specified. These specifications allow for an optimized preparation of the cells during forward or backward operation of the centrifuge, as well as for optimized conditions for a subsequent cell cultivation and/or bioproduction.

Various embodiments are directed towards options for automation by providing a valve arrangement. The valve arrangement serves for activating/deactivating the liquid lines of the liquid network in order to support the execution of the respective task.

Various embodiments define, that the centrifuge and/or the liquid pumping arrangement and/or the valve arrangement can be controlled by the electronic process control. The electronic process control can operate on a control software, which makes operation particularly flexible.

According to various embodiments, the first and/or second receptacle are providing as bio-reactor(s). Here it shows that the proposed solution is applicable for a vast number of different laboratory setups.

various embodiments are directed to a sensor arrangement comprising a biomass sensor in the recycling line. The sensor data of the biomass sensor define, when and where the liquid is transferred by the electronic process control to the second culture environment.

Various embodiments are directed to a bioprocess installation with an electronic process control and at least one bioprocess unit. The liquid network of the bioprocess installation comprises a recycling line enabling for a transfer of discharged cells into a second culture environment. All explanations given with regard to the first teaching are fully applicable to this second teaching.

According to various embodiments, the electronic process control can be able to perform the method by controlling the centrifuge and/or the liquid pumping arrangement and/or the valve arrangement, while some embodiments are directed to centrifuge specifications, enabling for an efficient design of the centrifuge.

Various embodiments are directed to an electronic process control of the proposed bioprocess installation. The electronic process control is designed to perform the proposed method by controlling the centrifuge and/or the liquid pumping arrangement and/or the valve arrangement. All explanations given with regard to the first and second teachings are fully applicable to this third teaching.

Various embodiments are directed to the use of a pre-mounted liquid network product, comprising a structure of interconnected liquid lines, as at least part of the liquid network of the proposed bioprocess installation. The premounting of the liquid network makes it particularly easy to quickly enable the bioprocess installation for operation. Moreover, the resulting ex-changeability of the liquid network product as a whole makes it easy to provide sterile conditions. This is particularly interesting for the recycling line in view of ensuring predetermined conditions for the subsequent cell cultivation.

In various embodiments, the structure of the interconnected liquid lines of the pre-mounted liquid network product can be designed as a single-use component, hence guaranteeing a cost-efficient process and at the same time perfectly sterile conditions. Again, all explanations given with regard to the first, second and third teaching are fully applicable to this fourth teaching.

Various embodiments provide a method for operating a bioprocess installation with an electronic process control and at least one bioprocess unit, wherein the bioprocess unit comprises a cell broth source with a first receptacle for cell broth including cultivation media and cells, establishing a culture environment for cell cultivation and/or bioproduction, wherein the bioprocess unit comprises a clarification setup with a centrifuge for the clarification of the cell broth by centrifugation, with a liquid pumping arrangement assigned to the centrifuge and with a liquid network with a number of liquid lines communicating with the liquid pumping arrangement, wherein out of a first culture environment established by the first receptacle, the cell broth is transferred to the centrifuge via the liquid network, which centrifuge is operated in a forward operation for cell separation and/or cell washing and a backward operation for cell discharging, wherein the liquid network comprises a recycling line and that in the backward operation, at least part of the discharged cells is being transferred into a second culture environment different from the first culture environment via the recycling line for subsequent cell cultivation and/or bioproduction.

In various embodiments, the second culture environment differs from the first culture environment with respect to the structural entity establishing the respective culture environment, and/or, that the second culture environment differs from the first culture environment with respect to liquid properties, and/or, that the second culture environment differs from the first culture environment with respect to culture environment conditions.

In various embodiments, the second culture environment provides culture environment conditions thereby favouring cell growth and/or bioproduction, and/or, that the second culture environment is the first culture environment, after having exchanged the cultivation media.

In various embodiments, the second culture environment is provided by the first receptacle, which, for establishing the second culture environment, is being brought to a cell-free and/or a liquid-free state in a preparation phase, such as, that the preparation phase consists of at least one workflow step to establish conditions for cell cultivation and/or bioproduction and that this workflow step can be at least one out of the group of media preparation, emptying, cleaning, maintaining, sterilizing, replacing and/or loading the first receptacle with media, such as rich media.

In various embodiments, the second culture environment is provided by a second receptacle, which is separate from the first receptacle, such as, that the second receptacle is free from cell broth of the first receptacle.

In various embodiments, the first receptacle and/or the second receptacle comprise(s) a feed line for a controlled feeding of media during cultivation according to a predefined feeding profile, and/or, that the second receptacle is a passive receptacle without a feed line and/or without electronic process control.

In various embodiments, the cell broth in the second receptacle is transferred into the first receptacle after an intermediate phase, such as, that the cells are kept in the intermediate phase until the cells reach a predefined vitality parameter, such as viability, wherein the predefined vitality parameter deviates less than 10% from the vitality parameter in the first culture environment.

In various embodiments, the cell broth in the second receptacle is transferred into the first receptacle not before a termination condition has been fulfilled with respect to the liquid level in the first receptacle.

In various embodiments, the bioprocess installation comprises two or more bioprocess units and that the second receptacle of each bioprocess unit is the first receptacle of a subsequent bioprocess unit such that the bioprocess units are cascaded.

In various embodiments, in the backward operation, after transferring at least part of the discharged cells into the second culture environment, a startup phase is initiated in the second culture environment to establish conditions for cell cultivation and/or bioproduction, such as, that in the startup phase, the discharged cells are grown to a cultivation stage used for bioproduction, in particular the exponential phase.

In various embodiments, in forward operation for cell washing, washing liquid, in some embodiments buffer or cultivation media, is transferred to the centrifuge via the liquid network, in some embodiments, that the washing liquid is tempered to a predetermined temperature, further, that the predetermined temperature can be in fixed relation to the temperature of the cell broth source.

In various embodiments, in backward operation for cell discharging, discharging liquid, in some embodiments buffer or cultivation media, is transferred to the centrifuge via the liquid network, such as, that the discharging liquid is tempered to a predetermined temperature, further, that the predetermined temperature can be in fixed relation to the temperature of the cell broth source.

In various embodiments, the bioprocess installation comprises a valve arrangement, which allows to deactivate and activate at least one of the liquid lines.

In various embodiments, the centrifuge and/or the liquid pumping arrangement and/or the valve arrangement are controlled by the electronic process control, such as, that the bioprocess installation comprises a sensor arrangement for measuring properties of the liquid in at least one of the receptacles and/or in at least one of the liquid lines, which sensor arrangement provides sensor signals to the electronic process control, such as, that the sensor arrangement measures at least one parameter out of the group of carbon-source concentration, nitrogen-source concentration, amino acids concentration, growth factors concentration, pH, temperature, oxygen concentration, carbon dioxide concentration, conductivity, pressure, biomass concentration, biomass production rate, product concentration, productivity and/or oxygen uptake rate.

In various embodiments, the first receptacle and/or the second receptacle is/are provided as a bioreactor, and/or, that a bioreactor process control serves for monitoring and/or controlling at least one parameter in the first receptacle and/or the second receptacle out of the group of carbon-source concentration, nitrogen-source concentration, amino acid concentration, growth factor concentration, oxygen concentration, carbon dioxide concentration, pH, temperature, conductivity, pressure, biomass concentration, biomass production rate, product concentration, productivity, oxygen uptake rate and/or stirring speed.

In various embodiments, the sensor arrangement comprises a biomass sensor in the recycling line, and that, in the backward operation of the centrifuge, depending on the sensor data of the biomass sensor, the liquid is transferred to the second culture environment or the waste receptacle by the electronic process control switching the valve arrangement accordingly.

Various embodiments provide a bioprocess installation with an electronic process control and at least one bioprocess unit, wherein the bioprocess unit comprises a cell broth source with a first receptacle for cell broth including cultivation media and cells, establishing a culture environment for cell cultivation and/or bioproduction, wherein the bioprocess unit comprises a clarification setup with a centrifuge for the clarification of the cell broth by centrifugation, with a liquid pumping arrangement assigned to the centrifuge and with a liquid network with a number of liquid lines communicating with the liquid pumping arrangement, wherein out of a first culture environment established by the first receptacle, the cell broth is transferred to the centrifuge via the liquid network, which centrifuge is operated in a forward operation for cell separation and/or cell washing and a backward operation for cell discharging, wherein the bioprocess installation comprises a valve arrangement, which allows to deactivate and activate at least one of the liquid lines, wherein the liquid network comprises a recycling line and that in the backward operation, at least part of the discharged cells may be transferred into a second culture environment different from the first culture environment via the recycling line for subsequent cell cultivation and/or bioproduction.

In various embodiments, the electronic process control is designed for performing the method as described herein by controlling the centrifuge and/or the liquid pumping arrangement and/or the valve arrangement.

In various embodiments, the centrifuge comprises at least one centrifuge chamber with a chamber inlet and a chamber outlet, in some embodiments, that the centrifuge comprises a centrifuge chamber, which is designed as a single-use component, and/or, that the recycling line is designed as a single-use component.

Various embodiments provide an electronic process control of the bioprocess installation as described herein, wherein the electronic process control is designed for performing the method as described herein by controlling the centrifuge and/or the liquid pumping arrangement and/or the valve arrangement.

Various embodiments provide a use of a pre-mounted liquid network product, which comprises a structure of interconnected liquid lines, as at least part of the liquid network of the bioprocess installation as described herein.

In various embodiments, the structure of interconnected liquid lines of the pre-mounted liquid network product provides at least the recycling line of the at least one bioprocess unit.

In various embodiments, the structure of interconnected liquid lines of the pre-mounted liquid network product is designed as a single-use component, in some embodiments, that the pre-mounted liquid network product comprises a sterile packaging for sterile housing of the structure of interconnected liquid lines.

### BRIEF DESCRIPTION OF THE DRAWINGS

In the following, an embodiment is being described with regard to the drawings. In the drawings

FIG. 1 schematically shows a first exemplary embodiment of a proposed bioprocess installation, with which a proposed method is executable,

FIG. 2 schematically shows a second exemplary embodiment of a proposed bioprocess installation, with which a proposed method is executable,

FIG. 3 schematically shows a third exemplary embodiment of a proposed bioprocess installation, with which a proposed method is executable and

FIG. 4 schematically shows an enlarged view of a proposed clarification setup of the proposed bioprocess installation according to FIGS. 1 to 3.

### DETAILED DESCRIPTION

The proposed method for operating a bioprocess installation 1 can be assigned to the upstream and downstream processes of a bioprocess, processing a liquid in the form of a cell broth for cell cultivation and/or bioproduction.

The term “liquid” is to be understood in a broad sense. It includes not only a pure liquid as such, but also emulsions and suspensions, e.g. a heterogeneous mixture of at least two different liquids or a heterogeneous mixture of solid particles and liquid.

The term “cell broth” is a suspension of solid cells or cell debris in media and describes the entirety of cultivation medium and the respective organism cultured in the cultivation medium. Accordingly, the term “cell broth source” means any manufactured device or system capable of producing and/or storing cell broth.

The term “upstream process” involves all the steps related to cell bank, inoculum (seed train) development, media development, optimization of growth kinetics and the cultivation process itself as well as the corresponding in-process control. The harvest of cells can be seen as both, part of upstream- and part of downstream-processing.

The term “downstream process” involves all the steps related to the recovery and the purifi-cation of biosynthetic products, particularly biopharmaceuticals, from natural sources such as animal or plant tissue or cell broth, including the recycling of salvageable components and the proper treatment and disposal of waste.

In general, cultivation of cells is currently used for the production of biopharmaceuticals, in particular proteins, such as human insulin, growth factors, hormones or vaccine proteins, in particular antibodies, antibody derivates, or the like. The product may as well be non-biopharmaceuticals, such as enzymes for food processing, laundry detergent enzymes, biode-gradable plastics or biofuels. The focus of some embodiments is on biopharmaceutical products secreted by the cells into the supernatant, such as antibodies. Additionally or alternatively, the product can be the cells themselves, in particular stem cells or tissues, produced by tissue engineering.

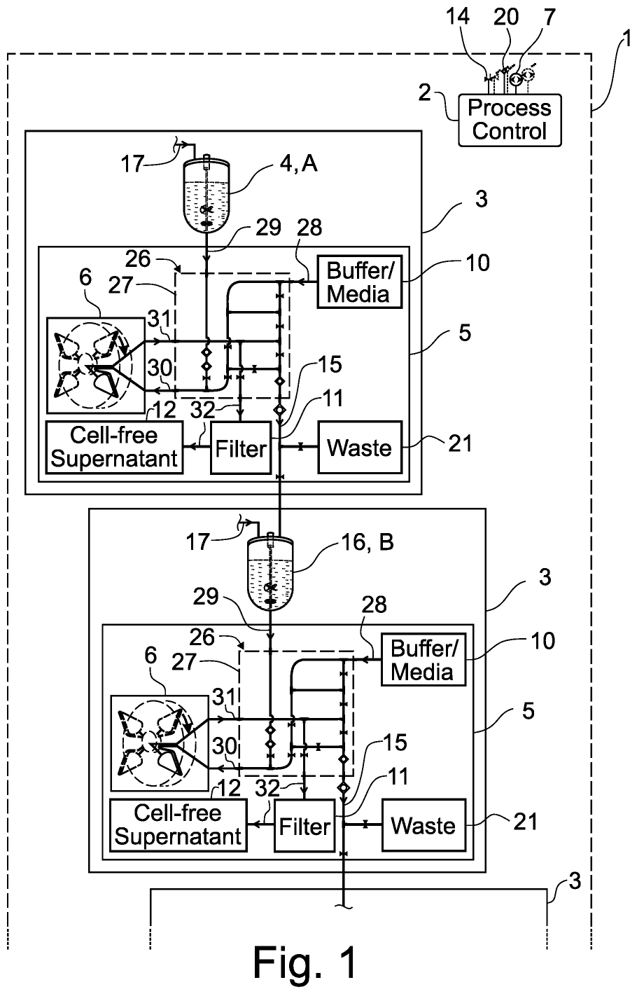

As shown in FIGS. 1 to 3, according to all embodiments the proposed method for operating a bioprocess installation 1 employs an electronic process control 2 and at least one bioprocess unit 3. The bioprocess unit 3 comprises a cell broth source with a first receptacle 4 for cell broth including cultivation media and cells. The term “cultivation media” means that the, in some embodiments microbial or eukaryotic, cells used for the bioprocess grow in or on spe-cially designed solid, semi-solid or liquid growth media, which supply the nutrients required by the respective organisms or cells. A variety of media exist, but invariably contain at least a carbon source, a nitrogen source, water, salts, and micronutrients.

Here, the respective culture environment A, B for cell cultivation and/or bioproduction is established by at least one physical, chemical and/or biological parameter, such as temperature, pH, carbon source concentration, nitrogen source concentration, amino acid source concentration, oxygen concentration, oxygen uptake rate, carbon dioxide concentration, media composition, type of media, type of receptacle, type of particles, in particular cells, cell concentration, cell viability, cell growth rate, cell productivity and/or biomass production rate.

Moreover, the bioprocess unit 3 comprises a clarification setup 5. This clarification setup 5 carries out a physical process using gravity to remove suspended solids from a liquid phase.

In general, the proposed clarification setup 5 can be used to separate any solid/liquid components from each other, including but not limited to cells and media.

For centrifugation, the clarification setup 5 comprises a centrifuge 6 for the clarification of the cell broth by centrifugation. “Centrifugation” is a term for sedimentation of particles in an artificially, by centrifugal forces created, gravitational field, wherein a significant reduction of separation time is achieved via large accelerating forces.

Here, the centrifuge 6 is designed as a fluidized bed centrifuge for performing a continuous centrifugation process. Various setups of the centrifuge 6 are described in EP 2 485 846 A1, the contents of which are hereby incorporated by reference herein.

The clarification setup 5 comprises a liquid pumping arrangement 7 assigned to the centrifuge 6 and a liquid network 8 with a number of liquid lines 9 communicating with the liquid pumping arrangement 7. Out of a first culture environment A established by the first receptacle 4, the cell broth is transferred to the centrifuge 6 via the liquid network 8.

The centrifuge 6 comprises a rotor, which may be rotated around the centrifuge rotor axis by an electric motor. For centrifugation, the liquid pumping arrangement 7 pumps cell broth to the centrifuge 6.

The centrifuge revolution speed, as well as the pumping rate, are, here, adjustable by the electronic process control 2, with the aim to establish a fluidized bed of particles, such as cells or cell debris, in the centrifuge 6. A fluidized bed is achieved when the centrifugal force on a particle is equal to the opposing fluid flow force so that a zero net force is exerted on the particle.

The centrifuge 6 can be operated in a forward operation for cell separation and/or cell washing. “Forward operation” means one out of two possible rotational directions of a centrifuge and describes the operation leading to a separation of liquid and solid particles, such as media and cells. This separation allows, on the one hand, a washing of separated cells with buffer, such as PBS buffer, or media, such as cultivation media, further such as rich media, coming from the buffer/media receptacle 10, and/or, on the other hand, the clarification of the supernatant. The goal here is to clarify the liquid supernatant from solid particles such as cells, cell debris, etc., which solid particles are considered biomass. The product to be obtained in this forward operation is the supernatant of the cell broth containing a product of interest, e.g. a recombinant protein, in particular an antibody.

The term “rich media” describes media that comprise higher concentrations of vitamins, growth factors, carbon-source, nitrogen-source and/or amino acid concentrations or the like and, in some embodiments, allow the respective organism to grow at its maximum growth rate due to the optimized nutrient concentrations. Growth factors and trace nutrients are in-cluded in the media for organisms incapable of producing all of the vitamins they require. Yeast extract is a common source of micronutrients and vitamins for media. Inorganic nutrients, including trace elements such as iron, zinc, copper, manganese, molybdenum and cobalt are typically present in unrefined carbon and nitrogen sources but may have to be added when purified carbon and nitrogen sources are used. Hence, in rich media, such as lysogeny broth (LB), the limiting factor in proliferation is the organisms physiology and not the availability of nutrients in the environment and/or protection from toxins or the like.

In order to obtain a, in some embodiments, particle-free, such as a cell-free, supernatant via forward operation of the centrifuge 6, the now centrifuged cell broth is subsequently pumped through a filter arrangement 11 of the clarification setup 5. Downstream of the filter arrangement 11 is provided with a supernatant reception 12, in particular a supernatant vessel. It is generally possible, that the at least one filter of the filter arrangement 11 is activated and deactivated manually, for example by at least one manually controllable valve 13.

It is also possible, that no valve 13 is assigned to the filter arrangement 11, in which case, instead of deactivating part of the filter arrangement 11, the flushing of the filter arrangement 11 is being started manually. The term “flushing” means a pre-conditioning of filters by rinsing them with buffer before the centrifugation process is started. In addition, after the centrifugation process is finished, a post-flushing of the filters can also be utilized in some cases in order to flush out remaining product in the filters and filter lines into the supernatant reception 12.

Alternatively, the centrifuge 6 can be operated in a backward operation. “Backward operation” means the second out of two possible rotational directions of a centrifuge and describes the operation leading to a discharge of the separated solid particles, such as cells. The product to be obtained in backward operation are the cells in the cell broth.

It can be particularly essential for some embodiments, that the liquid network 8 comprises a recycling line 15. In backward operation of the centrifuge 6, at least part of the discharged cells is being transferred to a second culture environment B via the recycling line 15.

This second culture environment B is different from the first culture environment A and set up for a recovery of the discharged cells. Subsequently, the discharged cells are used for cell cultivation and/or bioproduction. The term “recovery” is to be understood in a broad sense. It includes not only the collection of the discharged cells and their concentration in one particular place but is also providing an environment that maintains, feeds and/or nourishes the discharged cells. These measures prepare the discharged cells for subsequent cell cultivation and/or bioproduction.

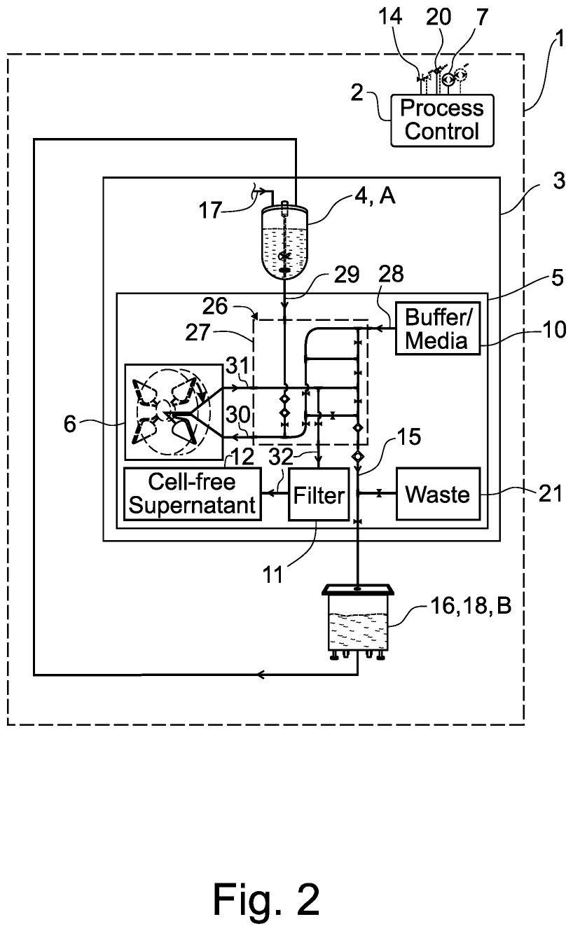

In the embodiments according to FIGS. 1 and 2, here, the second culture environment B differs from the first culture environment A with respect to the structural entity establishing the respective culture environment A, B. The entity establishing the second culture environment B can, for instance, be a second storage vessel, in particular a single-use storage and/or mixing bag, or a production vessel, such as a second bioreactor, in particular a single-use bioreactor bag.

In case of using a storage vessel, in particular a single-use storage and/or mixing bag, as structural entity establishing the culture environment B, the washed cell broth is collected in there, such as until the structural entity establishing the respective culture environment A is completely harvested. Hence, such a storage vessel enables a complete media exchange within the entity establishing the first culture environment A, without the risk of backmixing fresh and used cultivation media. This media exchange supports cell growth as well as productivity of the overall process. Moreover, a storage vessel, in particular a single-use storage and/or mixing bag, is significantly cheaper than a second bioreactor, in particular a sin-gle-use bioreactor bag, with its periphery equipment such as sensors, gassing, cooling or heating devices or the like, thereby increasing cost efficiency.

Additionally, the installation of a storage vessel, in particular of a single-use storage and/or mixing bag, requires much less time (several minutes) in comparison to the installation of a bioreactor, in particular of a single-use bioreactor bag (several hours). Moreover, any bioreactor, in particular any single-use bioreactor bag, requires elaborative integrity testing, elaborative connection to supply and exhaust lines, as well as elaborative calibration of the sensor arrangement 20 with its at least one sensor 19. After complete harvest and media exchange the cells can be transferred from the storage vessel, in particular from the single-use storage and/or mixing bag, back into the first culture environment A without the need of the above mentioned elaborative installation and calibration of an additional bioreactor, in particular of an additional single-use bioreactor bag.

Moreover, a storage vessel, in particular a single-use storage and/or mixing bag, requires only one inlet line and one outlet line for a sterile connection, whereas a bioreactor, in particular a single-use bioreactor bag, always requires a plurality of connections, in particular for feeding cultivation media, gas, antifoam, base for pH control or the like. Such a stream-lined process setup eliminates potential sources of errors, in particular user errors, e.g. calibration of sensors, sterility of connections or the like.

Conclusively, using a storage vessel, in particular a single-use storage and/or mixing bag, in contrast to using a second bioreactor, in particular a single-use bioreactor bag, as structural entity establishing the culture environment B, reduces the overall production costs, preparation times, as well as process complexity.

Additionally or alternatively, and as is provided by all embodiments in FIGS. 1 to 3, the second culture environment B differs from the first culture environment A with respect to liquid properties, e.g. choice of media, in particular choice of rich media, comprising i.a. optimized carbon-source concentrations, optimized nitrogen-source concentrations, optimized amino acid concentrations and/or optimized growth factor concentrations, and/or the liquid volume used in the bioprocess.

Further additionally or alternatively, and as is also provided by all embodiments in FIGS. 1 to 3, the second culture environment B differs from the first culture environment A with respect to culture environment conditions, such as choice of gas concentrations, in particular, oxygen and/or carbon dioxide concentrations, and/or choice of pH, and/or choice of temperature or the like. Exemplary amino acid-sources can be peptone or tryptone in concentrations of 0,5% to 2%. Exemplary carbon-sources can be glucose or sucrose in concentrations of 0,1% to 3%.

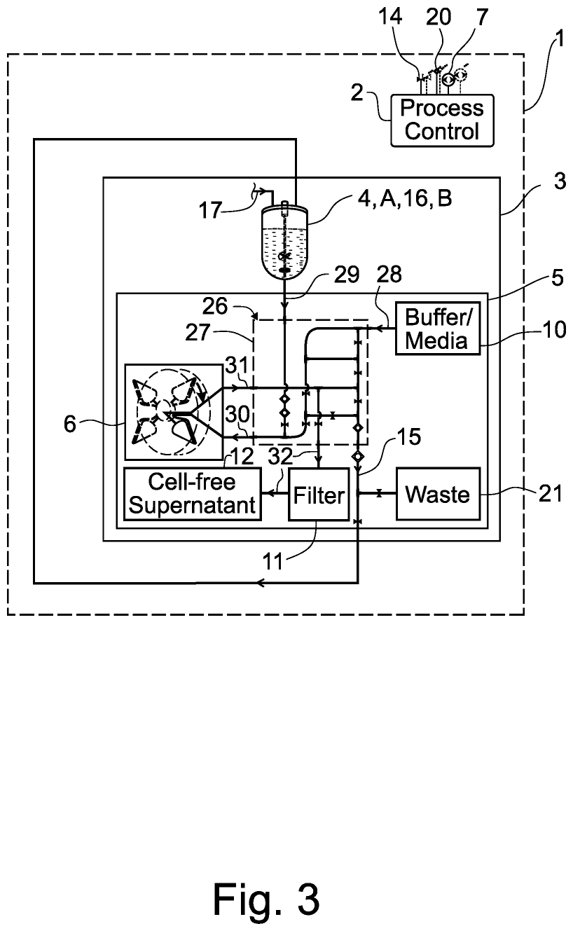

In the embodiment of FIG. 1, the further embodiment of FIG. 2, as well as in the yet further embodiment of FIG. 3, here, the second culture environment B is the first culture environment A, after having exchanged the cultivation media. In these embodiments, the discharged cells can be transferred for re-use of the cells for cell cultivation and/or bioproduction, after the cultivation media and/or cells have been, at least partially, removed. Thereby a backmixing of consumed and fresh cultivation media and/or cells is individually controllable.

In some embodiments, the resulting ratio of consumed to fresh liquid is individually adjustable. Additionally or alternatively, the second culture environment B can provide culture environment conditions thereby favouring cell growth and/or bioproduction. The term “Favouring cell growth and/or bioproduction” means an enhancement and support of at least one parameter either mirroring the cells' vitality and/or the cells' productivity, including but not limited to growth rate, viability, productivity, oxygen uptake rate and/or biomass production rate.

As shown in FIG. 3, in principle, the second culture environment B may be provided by the first receptacle 4, which, for establishing the second culture environment B, is being brought to a cell-free and/or a liquid-free state in a preparation phase. In various embodiments, the preparation phase consists of at least one workflow step to establish conditions for cell cultivation and/or bioproduction.

The above workflow step can be at least one out of the group of media preparation, emptying, cleaning, maintaining, sterilizing, replacing and/or loading the first receptacle with media, such as rich media. “Media preparation” describes the act of composing the media to be used in the bioprocess, in particular the calculations of the specific component ratios and the actual pouring of the media.

As another example, the workflow step may be at least partial, in some embodiments complete, emptying of the first receptacle 4. A cleaning step may be necessary, in particular, if the type of bioprocess or the product of interest has been changed. In this case, the first receptacle 4 needs to be at least partially, in some embodiments completely, cleaned. Another example may be the maintenance of the first receptacle 4, in order to ensure an optimal bioprocess, or a replacing of the first receptacle 4, in particular, if the receptacle 4, or in particular any receptacle of the bioprocess unit 3, is designed as a single-use device. Another possible workflow step could be the loading of the first receptacle 4 with media, in particular rich media, as described above. For this procedure, the media needs to be filled into the receptacle, first. Any cultivation media can be used, such as any rich media, that comprises higher nutrient concentrations etc. Further, these media do not constitute the limiting factor for cell cultivation and/or bioproduction. Here, and just as an example NutriStem® hPSC XF Medium, Gibco Cell Culture Medium, DMEM, IMDM, or the like might be used, depending on the bioprocess of choice. Further, media that lead to optimal cell growth and proliferation rates, healthy physiology, morphology and/or proper gene expression are used

In the further embodiments according to FIG. 1 and according to FIG. 2, the second culture environment B is provided by a second receptacle 16, which is separate from the first receptacle 4. This allows for a transfer of cells from the first receptacle 4 to a second receptacle 16. In some embodiments, when starting operation of the proposed bioprocess installation 1, the second receptacle 16 is free from cell broth of the first receptacle 4.

According to FIG. 1 and FIG. 2, the first receptacle 4 and/or the second receptacle 16 may comprise a feed line 17 for a controlled feeding of media during cultivation according to a predefined feeding profile. Such feeding profiles can be designed as pulse or continuous or a mixed feeding profile, wherein the feeding profile can be individually controllable and adjustable.

According to FIG. 1, the second receptacle 16 may be designed as a bioreactor. The term “bioreactor” in this context means any manufactured device or system that supports a bio-logically active environment by allowing monitoring and control of at least one parameter.

Additionally or alternatively, the second receptacle 16 is a passive receptacle 18 without a feed line 17 and/or without an electronic process control 2 (FIG. 2). As mentioned above, the second receptacle 16 can be designed as a storage vessel, in particular a single-use storage and/or mixing bag.

Hence, this second receptacle 16 can be designed as a passive receptacle 18 or an active receptacle, such as a bioreactor. “Passive receptacle” means a receptacle without a feed line 17 and/or without an electronic process control 2, which allows monitoring of at least one parameter within this passive receptacle 18. In contrast, “active receptacle” means a receptacle with a feed line 17 and/or with an electronic process control 2, hence allowing for monitoring and/or controlling of at least one parameter within this active receptacle.

In another embodiment, the cell broth in the second receptacle 16 can be transferred into the first receptacle 4 after an intermediate phase. “Intermediate phase” means a phase to prepare and/or nourish the cells until the cells are in a physiological state suitable for subsequent cell cultivation and/or bioproduction.

In various embodiments, the cells are kept in this intermediate phase until the cells reach a similar cultivation stage and/or similar vitality parameters present in the first culture environment A. In various embodiments, the cells are kept in this intermediate phase until the cells reach at least one predefined vitality parameter, including but not limited to viability, growth rate, productivity, oxygen uptake rate and/or biomass production rate. In another embodiment, the at least one predefined vitality parameter, in particular viability, deviates less than 10%, such as less than 5%, from the vitality parameter in the first culture.

In case the second receptacle 16 is a passive receptacle 18, as depicted in FIG. 2, the cell broth in the second receptacle 16 can be transferred to the first receptacle 4, not before a termination condition has been fulfilled with respect to the liquid level in the first receptacle 4. This means that, here in some embodiments, a specific liquid level in the first receptacle 4 must not be exceeded. The specific level can be individually defined according to the process re-quirements. The liquid level can be less than 10% of the total receptacle volume, further less than 5% of the total receptacle volume, further less than 1% of the total receptacle volume. Further the first receptacle 4 can be entirely empty.

According to FIG. 1, the bioprocess installation 1 can comprise two or more bioprocess units 3 and the second receptacle 16 of each bioprocess unit 3 is the first receptacle 4 of a subsequent bioprocess unit 3 such that the bioprocess units 3 are cascaded. The term “Cascaded” means here a consecutive sequence of bioprocess units 3 that follow one after another. Hence, the proposed method for operating a bioprocess installation 1 can be intended for employing one single receptacle or a multitude of receptacles, in particular bioreactors.

The proposed method can be used with any type of cell culture system, in particular bioreactors. The method can also be used with bioreactors of any size, in particular lab bioreactors or production bioreactors, and/or, the bioreactor(s) can be made of plastic, in particular bioplastic, glass or stainless steel, or can be designed as single-use bioreactor(s). Further, the method can be used with stationary or portable bioreactors. In another aspect, the method allows for bioreactors, wherein the cell viability is very high because there is a reduction in the stresses on cells.

As indicated by FIG. 1 and FIG. 2, in backward operation, after transferring at least part of the discharged cells into the second culture environment B, a startup phase can be initiated in the second culture environment B. The purpose of this startup phase is to establish conditions for cell cultivation and/or bioproduction and is initiated in the second culture environment B prior to cell cultivation and/or bioproduction. Exemplary conditions can be a cell viability of at least 80%, of at least 90%, or further of at least 95%. Further exemplary conditions can be a viable cell concentration of at least 1×10⁷ cells per milliliter, of at least 2×10⁷ cells per milliliter, of at least 3×10⁷ cells per milliliter, or of at least 4×10⁷ cells per milliliter. In various embodiments, in the startup phase, the discharged cells are grown to a cultivation stage used for cell proliferation and/or bioproduction, in particular the log phase.

“Cultivation stage” refers to the growth of cells in batch culture, which can be subdivided into four phases: Lag phase, log (also called “exponential”) phase, stationary phase and death phase. The cultivation stage can be any cultivation stage suitable for the bioprocess of interest. In various embodiments, the log phase or stationary phase. The term “log phase” or “exponential phase” refers to a growth period characterized by cell doubling per time unit. The term “stationary phase” describes a situation in which growth rate and death rate are equal, caused by a growth-limiting factor such as the depletion of an essential nutrient, and/or the formation of an inhibitory product such as an organic acid.

Proof of concept experiments show that various embodiments in an upstream process leads to an increase of approx. 33% in viable cell concentration and an increase of at least 47%, or of at least 56%, in product amount, here monoclonal antibody amount, while cultivation time could be reduced by one day, in comparison to a standard fed-batch process. In a downstream process, various embodiments can lead to an increased viable cell concentration of 4,2×10⁷ cells per milliliter, while a product recovery rate of at least 96% was achieved.

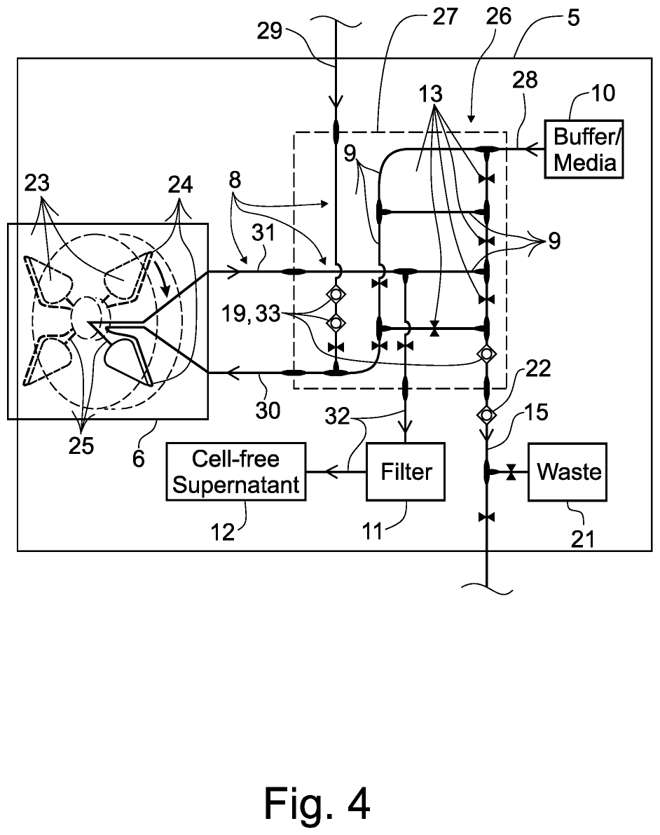

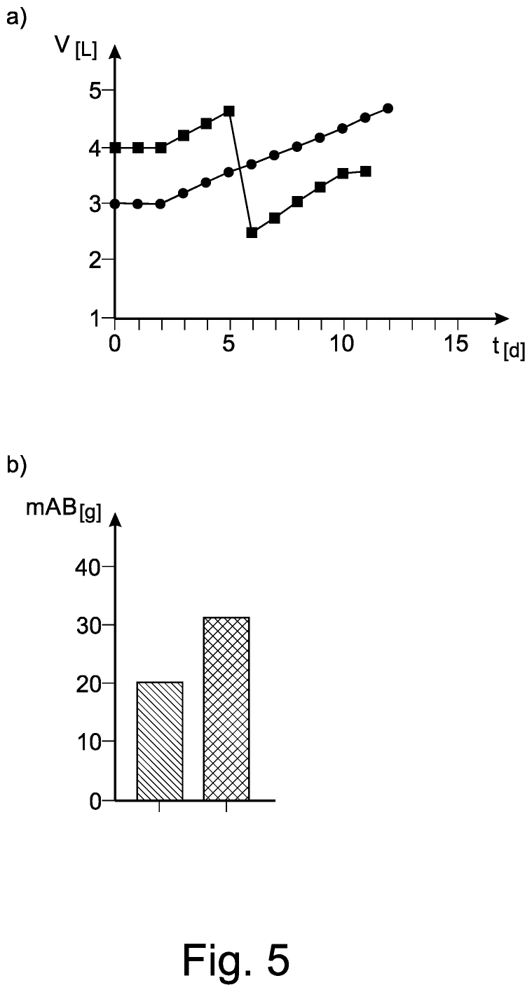

Here, in various embodiments according to FIGS. 1 to 3, in forward operation for cell washing, washing liquid, such as water, buffer or cultivation media, such as rich media, coming from the buffer/media receptacle 10, is transferred to the centrifuge 6 via the liquid network 8 for cell washing, as indicated by FIG. 4. The water, buffer or cultivation media, such as rich media, used as washing liquid, can be either fresh or used or a mixture of both. In various embodiments, the washing liquid is tempered to a predetermined temperature. In various embodiments, the predetermined temperature is in fixed relation to the temperature of the cell broth source. In various embodiments, the predetermined temperature can be adjusted according to the cells of choice, the bioprocess of choice and/or the product of choice. In various embodiments, the predetermined temperature is between 25° C. and 37° C., further such as, in case microbial or mammalian cells are used, between 35° C. and 37° C., and/or, in case of insect, fish, or amphibian cells are used, between 25° C. and 28° C.

Here, in all embodiments according to FIGS. 1 to 3, in backward operation for cell discharging, discharging liquid, such as water, buffer or cultivation media, such as rich media, coming from the buffer/media receptacle 10, is transferred to the centrifuge 6 via the liquid network 8 for cell discharging, as indicated by FIG. 4. The water, buffer or cultivation media, such as rich media, used as discharging liquid, can be either fresh or used or a mixture of both. In various embodiments, the discharging liquid is tempered to a predetermined temperature. In various embodiments, the predetermined temperature is in fixed relation to the temperature of the cell broth source. In various embodiments, the predetermined temperature can be adjusted according to the cells of choice, the bioprocess of choice and/or the product of choice. In various embodiments, the predetermined temperature is between 25° C. and 37° C., further, in case microbial or mammalian cells are used for the bioprocess, between 35° C. and 37° C., and/or, in case insect, fish, or amphibian cells are used, between 25° C. and 28° C. The washing liquid and the discharging liquid can either be different or the same liquid.

The bioprocess installation 1, here, comprises a valve arrangement 14 with at least one valve 13, that allows to deactivate and activate at least one of the liquid lines 9, such as by closing and opening the respective valve 13. Here, it is provided that the valve arrangement 14 is controlled by the electronic process control 2. The electronic process control 2 may activate or deactivate at least one of the liquid lines 9 via the valve arrangement 14 based on the sensor signals of the at least one sensor 19 of the sensor arrangement 20 and according to the respective control strategy.

Here, the term “deactivating” means, that liquid flow through the respective liquid line may be blocked by the respective valve. “Activating” means, accordingly, that liquid flow is al-lowed through the respective liquid line.

The valves 13 of the valve arrangement 14 are located within at least one of the respective liquid lines 9 and/or at one end of the respective liquid line(s) 9 to be activated and deactivated. The at least one valve 13 can be automatically selectively closed or opened during use. The individual valve(s) 13 of the valve arrangement 14 is/are controlled by the electronic process control 2.

The centrifuge 6 and/or the liquid pumping arrangement 7 and/or the valve arrangement 14 can be controlled by the electronic process control 2. The clarification setup 5, as can be seen in FIG. 4, here comprises a sensor arrangement 20 with at least one sensor 19 for measuring properties of the liquid in at least one of the receptacles and/or in at least one of the liquid lines 9, which sensor arrangement 20 provides sensor signals to a process control, such as the electronic process control 2. The process control of the sensor arrangement 20 may be designed as a centralized control unit, such as the electronic process control 2, or decentralized control unit. The sensor arrangement 20 provides sensor signals to the respective process control. In some embodiments, the sensor arrangement 20 measures at least one parameter out of the group of carbon-source concentration, nitrogen-source concentration, amino acids concentration, growth factors concentration, pH, temperature, oxygen concentration, carbon dioxide concentration, conductivity, pressure, biomass concentration, biomass production rate, product concentration, productivity and/or oxygen uptake rate.

In the embodiments according to FIGS. 1 to 3, the first receptacle 4 and/or the second receptacle 16 is/are provided as a bioreactor. Additionally or alternatively, a bioreactor process control, such as the centralized or decentralized electronic process control 2, serves for monitoring and/or controlling at least one parameter in the first receptacle 4 and/or the second receptacle 16 out of the group of carbon-source concentration, nitrogen-source concentration, amino acids concentration, growth factors concentration, oxygen concentration, carbon dioxide concentration, pH, temperature, conductivity, pressure, biomass concentration, biomass production rate, product concentration, productivity, oxygen uptake rate and/or stirring speed. The bioreactor process control can be designed as a digital control unit (DCU) and/or as a multi fermenter control system (MFCS), or the like.

The term “Monitoring” means measurement and/or documentation of the, at least one, parameter measured by the sensor arrangement 20. The term “Controlling” means measurement, documentation and/or control of the at least one parameter measured by the sensor arrangement 20, and/or, is designed with or without a feedback control. The term “Feedback control” means a self-regulation of the system's activities based on the measurement of the at least one sensor 19 of the sensor arrangement 20.

As indicated by FIG. 4, the sensor arrangement 20 can comprise at least one biomass sensor 22 in the recycling line 15 and the clarification setup 5 comprises a waste receptacle 21, such as designed as a waste vessel.

In various embodiments, the sensor arrangement 29 comprises a biomass sensor 22, such as an optical biomass sensor, in the recycling line 15 for measuring an occurrence level of biomass, such as the biomass concentration. During forward operation, depending on the measured occurrence level, such as biomass concentration, the electronic process control 2 then deactivates or activates the feed line 17 and/or starts the washing step.

As an alternative or in addition, the sensor arrangement 20 comprises a supernatant sensor, such as an optical supernatant sensor, for measuring an occurrence level of supernatant in the respective liquid line 9. During the forward operation, depending on the measured occurrence level of supernatant, here, the electronic process control 2 then deactivates or activates the respective liquid lines 9, such as the recycling line 15, via the valve arrangement 14.

Here, in the backward operation of the centrifuge 6, depending on the sensor data of the biomass sensor 22 in the recycling line 15, the liquid, such as the solid particles in discharging liquid, further the cells in discharging liquid, can be transferred to the second culture environment B or the waste receptacle 21 by the electronic process control 2 switching the valve arrangement 14 accordingly.

According to various embodiments, a bioprocess installation 1 is provided with an electronic process control 2 and at least one bioprocess unit 3. The bioprocess unit 3 comprises a cell broth source with a first receptacle 4 for cell broth including cultivation media and cells, establishing a culture environment A, B for cell cultivation and/or bioproduction. The bioprocess unit 3 further comprises a clarification setup 5 with a centrifuge 6 for the clarification of the cell broth by centrifugation, with a liquid pumping arrangement 7 assigned to the centrifuge 6 and with a liquid network 8 with a number of liquid lines 9 communicating with the liquid pumping arrangement 7, wherein out of a first culture environment A established by the first receptacle 4, the cell broth is transferred to the centrifuge 6 via the liquid network 8, which centrifuge 6 is operated in a forward operation for cell separation and/or cell washing and in a backward operation for cell discharging. Moreover, the bioprocess installation 1 comprises a valve arrangement 14, which allows to deactivate and activate at least one of the liquid lines 9. All explanations given before are fully applicable to this teaching.

It is essential, that the liquid network 8 comprises a recycling line 15 and that in the backward operation, at least part of the discharged cells may be transferred into a second culture environment B different from the first culture environment A via the recycling line 15 for subsequent cell cultivation and/or bioproduction.

The electronic process control 2 can be designed to perform the proposed method by controlling the centrifuge 6 and/or the liquid pumping arrangement 7 and/or the valve arrangement 14. The electronic process control 2 may be realized as a central unit controlling all or at least most of the components of the bioprocess installation 1. The electronic process control 2 may also be realized in a decentralized structure, comprising a number of decentralized units. In some embodiments, the at least one electronic process control(s) 2 direct(s) the opening and closing of the one or more valve 13, the flow rates of the one or more pump of the liquid pumping arrangement 7, the rotational speed of the rotor, either directly or via a motor, and/or the flow velocity of the fluid and/or particles from a cell broth source, such as a bioreactor.

Such an electronic process control 2 comprises for instance at least one digital control unit (DCU) and/or at least one multi fermenter control system (MFCS), which comprises a local processor unit and a local data storage itself. The MFCS also provides a centralized process management system, dispatching requests to the digital control unit. Additionally or alternatively, such an electronic process control 2 can comprise a computer, and/or a server, and/or a smartphone or the like. Here, the electronic process control 2 is individually adjustable and/or programmable and/or comprises at least one microprocessor, on which a software may be run.

As can be seen in the enlarged view in FIG. 4 showing of the clarification setup 5, here the centrifuge 6 can comprise at least one centrifuge chamber 23 with a chamber inlet 24 and a chamber outlet 25.

The expression “chamber inlet” means that the liquid to be centrifuged enters the, at least one centrifuge chamber 23 via the chamber inlet 24. The expression “chamber outlet” means that the centrifuged liquid exits the at least one centrifuge chamber 23 via the chamber outlet 25. This is only to be understood as a definition of the fluid interface of the centrifuge chamber 23. In case the forward operation is switched to the backward operation, the chamber inlet 24 may be used as chamber outlet 25 and the chamber outlet 25 may be used as chamber inlet 24, respectively.

In the beginning of a forward operation it may be necessary, that remaining buffer and/or remaining supernatant in the centrifuge chamber 23 is flushed into the waste receptacle 21 via the liquid network 8, before the chamber outlet 25 is connected by valve switching to the filter arrangement 11. It may also be necessary, that in the beginning of the washing step, buffer and remaining supernatant in the centrifuge chamber 23 is being pumped to the filter arrangement 11, before the chamber outlet 26 is connected by valve switching to the waste receptacle 23 via the recycling line 10.

Here, the electronic process control 2 is automatically executing those steps sequentially, according to a certain, individually definable control strategy. In the easiest case, the control strategy includes the execution of the respective operations according to a fixed sequence in a fixed time pattern. However, the control strategy may well be based on sensor signals.

Generally, it may be provided, that the centrifuge 6 comprises a centrifuge chamber 23, which is designed as a single-use component, and/or, that the recycling line 15 is designed as a single-use component. As an alternative or in addition, it may be provided, that at least part of the liquid network 8 is designed as a single-use component. Further in some embodiments, at least part of the filter arrangement 11 is designed as a single-use component. The single-use component can be made of plastic material at least partly, such that it may be realized with low effort. In various embodiments, the single-use component is at least partly made of a silicon material and/or polymer material and/or bioplastic.

According to various embodiments, an electronic process control of the bioprocess installation 1 is provided. Again, reference is made to all explanations given before.

It is essential, that the electronic process control 2 is designed for performing the proposed method by controlling the centrifuge 6 and/or the liquid pumping arrangement 7 and/or the valve arrangement 14.

According to various embodiments, the use of a pre-mounted liquid network product 26, which comprises a structure of interconnected liquid lines 27, as at least part of the liquid network 8 of the proposed bioprocess installation 1, is provided. Again, all explanations given before are fully applicable.

Here, and as can be seen in FIGS. 1 to 3, the pre-mounted liquid network product 26 can be designed as two separate, in some embodiments different, pre-mounted liquid network products 26, comprising two separate, in some embodiments different, structures of interconnected liquid lines 27. One pre-mounted liquid network product 26 can be designed to support its use for supernatant clarification in the forward operation of the centrifuge 6 by providing at least one liquid line 9 of the liquid network 8, optimized for supernatant clarification, such as optimized regarding a minimal liquid line distance, liquid network 8 and/or liquid lines 9. The second pre-mounted liquid network product 26 is designed to support its use for separation of cells and liquid in the backward operation of the centrifuge 6 by providing a liquid network 8 and liquid lines 9, optimized for the separation of cells and liquid, such as optimized regarding a minimal liquid line distance, liquid network 8 and/or liquid lines 9.

Here, such a pre-mounted liquid network product 26 comprises at least one inlet buffer/media line 28, designed for a supply with buffer or media, one inlet cell broth line 29, designed for a supply with cell broth by the cell broth source, one inlet centrifuge line 30, designed for a supply with centrifuged cell broth via the chamber outlet 25, one outlet centrifuge line 31, designed for an outflow of liquid into the chamber inlet, one outlet supernatant line 32, designed for an outflow of supernatant towards the filter arrangement 11 and the supernatant reception 12 and one recycling line 15, designed for an outflow of liquid, in particular, discharged cells in discharging liquid, towards the waste receptacle 21 or the second culture environment B.

During the discharging step, the buffer may then be pumped from the buffer/media receptacle 10 to the chamber outlet 25 via an inlet buffer/media line 28, while the buffer or media including solid particles, such as cell harvest, is flowing from the chamber inlet 24 towards the recycling line 15. During the discharging step, the liquid pressure and/or the liquid flow in the inlet buffer/media line 28 may be controlled by the liquid pumping arrangement 7.

In particular, the outlet supernatant line 32 as such may be provided as a single-use component. With this, it is particularly easy to guarantee the liquid tightness of the outlet supernatant line 32 without the risk of compromising the liquid tightness due to user errors during installation.

Here it is to be understood, that some of the liquid lines 9 may overlap, such that a respective liquid flow, originally assigned to a specific liquid line 9, may double-use at least one of the liquid lines 9, originally assigned to another liquid flow, at least along a certain liquid line 9 section. Accordingly, the respective liquid lines 9 do not have to be separate from each other along their complete extent. This is true for all other definitions of liquid lines 9 presented here and being part of the liquid network 8, which are each being provided by part of the liquid network 8.

Additionally or alternatively, the pre-mounted liquid network product comprises at least one, in some embodiments at least three, in some embodiments exactly three, bubble sensor(s) 33. In order to distinguish liquid from the air, at least one of the bubble sensors 33 can be designed as an optical sensor and/or at least one of the bubble sensors 33 can be designed as ultrasonic sensor and/or at least one of the bubble sensors 33 can be designed as conductivity sensor. Depending on the sensor data of the at least one bubble sensor 33, the liquid is transferred to the respective liquid line(s) 9 and/or to the respective receptacle of interest.

In various embodiments, in the enlarged view of the clarification setup 5 according to FIG. 4, the structure of interconnected liquid lines 27 of the pre-mounted liquid network product 26 provides at least the recycling line 15 of the at least one bioprocess unit 3.

In various embodiments, the structure of interconnected liquid lines 27 of the pre-mounted liquid network product 26 is designed as a single-use component. All in all, it may be advan-tageous to provide all components, that are in direct contact with liquid, as single-use components. This would at least include all liquid lines 7, the at least one centrifuge chamber 23, as well as all filters of the filter arrangement 11. It may also include at least part of the valve arrangement 14 and/or at least part of the sensor arrangement 20. In particular, single-use sensors may be part of the sensor arrangement 20. Here, the pre-mounted liquid network product 26 comprises a sterile packaging for sterile housing of the structure of interconnected liquid lines 27.

1. A method for operating a bioprocess installation with an electronic process control and at least one bioprocess unit, wherein the bioprocess unit comprises a cell broth source with a first receptacle for cell broth including cultivation media and cells, establishing a culture environment for cell cultivation and/or bioproduction, wherein the bioprocess unit comprises a clarification setup with a centrifuge for the clarification of the cell broth by centrifugation, with a liquid pumping arrangement assigned to the centrifuge and with a liquid network with a number of liquid lines communicating with the liquid pumping arrangement,
wherein out of a first culture environment established by the first receptacle, the cell broth is transferred to the centrifuge via the liquid network, which centrifuge is operated in a forward operation for cell separation and/or cell washing and a backward operation for cell discharging,
wherein the liquid network comprises a recycling line and that in the backward operation, at least part of the discharged cells is being transferred into a second culture environment different from the first culture environment via the recycling line for subsequent cell cultivation and/or bioproduction.

2. The method according to claim 1, wherein the second culture environment differs from the first culture environment with respect to the structural entity establishing the respective culture environment, and/or, that the second culture environment differs from the first culture environment with respect to liquid properties, and/or, that the second culture environment differs from the first culture environment with respect to culture environment conditions.

3. The method according to claim 1, wherein the second culture environment provides culture environment conditions thereby favouring cell growth and/or bioproduction, and/or, that the second culture environment is the first culture environment, after having exchanged the cultivation media.

4. The method according to claim 1, wherein the second culture environment is provided by the first receptacle, which, for establishing the second culture environment, is being brought to a cell-free and/or a liquid-free state in a preparation phase.

5. The method according to claim 1, wherein the second culture environment is provided by a second receptacle, which is separate from the first receptacle.

6. The method according to claim 1, wherein the first receptacle and/or the second receptacle comprise(s) a feed line for a controlled feeding of media during cultivation according to a predefined feeding profile, and/or, that the second receptacle is a passive receptacle without a feed line and/or without electronic process control.

7. The method according to claim 5, wherein the cell broth in the second receptacle is transferred into the first receptacle after an intermediate phase.

8. The method according to claim 5, wherein the cell broth in the second receptacle is transferred into the first receptacle not before a termination condition has been fulfilled with respect to the liquid level in the first receptacle.

9. The method according to claim 1, wherein the bioprocess installation comprises two or more bioprocess units and that the second receptacle of each bioprocess unit is the first receptacle of a subsequent bioprocess unit such that the bioprocess units are cascaded.

10. The method according to claim 1, wherein in the backward operation, after transferring at least part of the discharged cells into the second culture environment, a startup phase is initiated in the second culture environment to establish conditions for cell cultivation and/or bioproduction.

11. The method according to claim 1, wherein in forward operation for cell washing, washing liquid is transferred to the centrifuge via the liquid network.

12. The method according to claim 1, wherein in backward operation for cell discharging, discharging liquid is transferred to the centrifuge via the liquid network.

13. The method according to claim 1, wherein the bioprocess installation comprises a valve arrangement, which allows to deactivate and activate at least one of the liquid lines.

14. The method according to claim 1, wherein the centrifuge and/or the liquid pumping arrangement and/or the valve arrangement are controlled by the electronic process control.

15. The method according to claim 1, wherein the first receptacle and/or the second receptacle is/are provided as a bioreactor, and/or, that a bioreactor process control serves for monitoring and/or controlling at least one parameter in the first receptacle and/or the second receptacle out of the group of carbon-source concentration, nitrogen-source concentration, amino acid concentration, growth factor concentration, oxygen concentration, carbon dioxide concentration, pH, temperature, conductivity, pressure, biomass concentration, biomass production rate, product concentration, productivity, oxygen uptake rate and/or stirring speed.

16. The method according to claim 1, wherein the sensor arrangement comprises a biomass sensor in the recycling line, and that, in the backward operation of the centrifuge, depending on the sensor data of the biomass sensor, the liquid is transferred to the second culture environment or the waste receptacle by the electronic process control switching the valve arrangement accordingly.

17. A bioprocess installation with an electronic process control and at least one bioprocess unit, wherein the bioprocess unit comprises a cell broth source with a first receptacle for cell broth including cultivation media and cells, establishing a culture environment for cell cultivation and/or bioproduction, wherein the bioprocess unit comprises a clarification setup with a centrifuge for the clarification of the cell broth by centrifugation, with a liquid pumping arrangement assigned to the centrifuge and with a liquid network with a number of liquid lines communicating with the liquid pumping arrangement, wherein out of a first culture environment established by the first receptacle, the cell broth is transferred to the centrifuge via the liquid network, which centrifuge is operated in a forward operation for cell separation and/or cell washing and a backward operation for cell discharging,
wherein the bioprocess installation comprises a valve arrangement, which allows to deactivate and activate at least one of the liquid lines,
wherein the liquid network comprises a recycling line and that in the backward operation, at least part of the discharged cells may be transferred into a second culture environment different from the first culture environment via the recycling line for subsequent cell cultivation and/or bioproduction.

18. The bioprocess installation according to claim 17, wherein the electronic process control is designed for controlling the centrifuge and/or the liquid pumping arrangement and/or the valve arrangement.

19. The bioprocess installation according to claim 17, wherein that the centrifuge comprises at least one centrifuge chamber with a chamber inlet and a chamber outlet, preferably, that the centrifuge comprises a centrifuge chamber, which is designed as a single use component, and/or, that the recycling line is designed as a single use component.

20. The electronic process control of the bioprocess installation according to claim 17, wherein the electronic process control is designed for controlling the centrifuge and/or the liquid pumping arrangement and/or the valve arrangement.

21. The use of a pre-mounted liquid network product, which comprises a structure of interconnected liquid lines, as at least part of the liquid network of the bioprocess installation according to claim 17.

22. The use according to claim 21, wherein the structure of interconnected liquid lines of the pre-mounted liquid network product provides at least the recycling line of the at least one bioprocess unit.

23. The use according to claim 21, wherein the structure of interconnected liquid lines of the pre-mounted liquid network product is designed as a single-use component.
